(19) United States
(12) Patent Application Publication
(10) Pub. No.: US 2013/0189338 A1
(43) Pub. Date: Jul. 25, 2013

(54) BONE FIBERS HAVING EXTENDED LENGTH

(71) Applicants: Susan J. Drapeau, Cordova, TN (US); Guobao Wei, Milltown, NJ (US)

(72) Inventors: Susan J. Drapeau, Cordova, TN (US); Guobao Wei, Milltown, NJ (US)

(73) Assignee: Warsaw Orthopedic, Inc., Warsaw, IN (US)

(21) Appl. No.: 13/353,393

(22) Filed: Jan. 19, 2012

(51) Int. Cl. A61K 35/32; A61K 38/43; A61K 39/00; A61K 38/20; A61P 19/08; A61P 7/04; A61P 31/12; A61P 9/00; A61P 35/00; A61P 37/06; A61P 37/04; A61K 9/00; A61P 31/00

(52) U.S. Cl. 424/402; 424/549; 424/484; 424/93.7; 424/94.1; 424/400; 424/184.1; 424/85.2; 424/529

(57) ABSTRACT

Demineralized bone matrix fibers and demineralized bone compositions are provided. Implantable demineralized bone matrix compositions include elongated bone fibers having an average length from about 2 cm to about 6 cm and an aspect ratio from about 50:1 to about 1000:1. The demineralized bone matrix compositions also include a carrier in an amount sufficient to produce a cohesive formable mass. The elongated fibers can easily entangle to provide an improved demineralized bone matrix having increased osteoconductivity.

### TECHNICAL FIELD

The present disclosure generally relates to a surgical bone product useful in bone repair and replacement. More specifically, the present disclosure relates to an implantable bone composition including bone fibers having extended length.

### BACKGROUND

The rapid and effective repair of bone defects caused by injury, disease, wounds, or surgery is a goal of orthopedic surgery. Toward this end, a number of compositions and materials have been used or proposed for use in the repair of bone defects. The biological, physical, and mechanical properties of the compositions and materials are among the major factors influencing their suitability and performance in various orthopedic applications.

Autologous cancellous bone (ACB), also known as autograft or autogenous bone, is considered the gold standard for bone grafts. ACB is osteoinductive and nonimmunogenic, and, by definition, has all of the appropriate structural and functional characteristics appropriate for the particular recipient. Unfortunately, ACB is only available in a limited number of circumstances. Some individuals lack ACB of appropriate dimensions and quality for transplantation, and donor site pain and morbidity can pose serious problems for patients and their physicians.

Much effort has been invested in the identification or development of alternative bone graft materials. Demineralized bone matrix (DBM) implants have been reported to be particularly useful. Demineralized bone matrix is typically derived from cadavers. The bone is removed aseptically and/or treated to kill any infectious agents. The bone is then particulated by milling or grinding and then the mineral components are extracted for example, by soaking the bone in an acidic solution.

The acid treatment dissolves inorganic mineral components and acid-soluble proteins in the bone, leaving behind a collagen matrix as well as acid-insoluble proteins and growth factors. Among the remaining acid-insoluble proteins and growth factors are bone morphogenic proteins (BMPS) and transforming growth factors (TGFs). DBM is a desirable component of bone graft materials because it provides an osteoinductive matrix and exhibits osteoconductive potential, thereby promoting bone growth and healings. Moreover, DBM is fully resorbable, and bone graft materials containing organic DBM are highly biocompatible because it contains many of the components of natural bone. Advantageously, DBM costs less than many other available organic bone composition additives, such as isolated BMPs.

After acid treatment the remaining matrix is malleable and can be further processed and/or formed and shaped for implantation into a particular site in the recipient. Demineralized bone prepared in this manner contains a variety of components including proteins, glycoproteins, growth factors, and proteoglycans. Following implantation, the presence of DBM induces cellular recruitment to the site of injury. The recruited cells may eventually differentiate into bone forming cells. Such recruitment of cells leads to an increase in the rate of wound healing and, therefore, to faster recovery for the patient.

Current DBM formulations have various drawbacks. First, while the collagen-based matrix of DBM is relatively stable, the active factors within the DBM matrix are rapidly degraded. The osteogenic activity of the DBM may be significantly degraded within 24 hours after implantation, and in some instances the osteogenic activity may be inactivated within 6 hours. Therefore, the factors associated with the DBM are only available to recruit cells to the site of injury for a short time after transplantation. For much of the healing process, which may take weeks to months, the implanted material may provide little or no assistance in recruiting cells.

The vast majority of the DBM particles possess random, irregular geometries with bone particles size ranging from about 110 to 850 microns. The combination of the glycerol's high water solubility and reduced viscosity causes the composition to be “runny” and to flow away from the site almost immediately after placement, thus preventing the proper retention of the composition at the implant site. In order to address the lack of cohesiveness of DBM at implant sites, some binders, such as high molecular weight hydrogels or other polymers as carrier vehicles have been utilized. However, these binders can negatively affect the biocompatibility and osteoinductivity of the DBM composition. Furthermore, these binders provide cohesiveness to the composition only prior to its implantation; following implantation, these binders are eroded or dissolved from the implant site and, consequently, the implant does not retain its shape in vivo.

It is, therefore, desirable to provide fiber-based demineralized bone matrices for implantation that exhibits improvements in key mechanical properties, including cohesiveness, fiber length, fiber diameter or width, fiber aspect ratio, or a combination of multiple variables.

### SUMMARY

Elongated demineralized bone fibers having an average length greater than 2 cm are provided. In some embodiments, the elongated fibers have an average length from about 2.1 cm to about 6 cm.

In various embodiments, the average length of the elongated demineralized bone fibers is greater than the average width. In various embodiments the aspect ratio of the elongated demineralized bone fibers is from about 50:1 to about 1000:1.

In some embodiments the elongated bone fibers are obtained from cortical autogenic, cortical allogenic, cortical xenogenic cancellous autogenic, cancellous allogenic, cancellous xenogenic, cortical transgenic, cancellous transgenic, corticocancellous autogenic, corticocancellous allogenic, corticocancellous xenogenic or corticocancelldus transgenic bone.

In other embodiments an implantable composition comprising elongated demineralized bone fibers having an average length from about 2.1 cm to about 6 cm are provided. The implantable composition can include other additives, such as for example, collagen, collagen derivatives, antiviricides, antimicrobials, antibiotics, biocidal sugars, amino acids, peptides, vitamins, inorganic elements, co-factors for protein synthesis, hormones, endocrine tissue, endocrine tissue fragments, enzymes, polymer cell scaffolds with parenchymal cells, stem cells, angiogenic drugs, collagen lattices, antigenic agents, cytoskeletal agents, cartilage fragments, living cells including stem cells, natural extracts, tissue transplants, demineralized bone powder, autogenous tissues, bioadhesives, bone morphogenic proteins (BMPs), angiogenic factors, transforming growth factor (TGF-beta), insulin-like growth factor (IGF-1), growth hormones, bone digestors, antitumor agents, immuno-suppressants, permeation enhancers, enamine derivatives, nucleic acids, or combinations thereof.

In some embodiments the implantable composition further includes at least one radiopaque material comprising barium sulfate, iodine containing compounds, titanium, mineralized bone or mixtures thereof.

Additives containing autograft bone marrow aspirate, autograft bone, preparations of selected autograft cells, autograft cells containing genes encoding bone promoting action, autograft cells expanded outside the body and returned or combinations thereof can also be included in the implantable composition.

The present application also provides a demineralized bone matrix composition including an elongated demineralized bone fiber having an average length greater than 2 cm and, in some embodiments from about 2.0 cm to about 6 cm and a carrier sufficient to produce a cohesive formable mass. In certain embodiments, more than 90% of the cohesive formable mass retains its initial shape dimension in an aqueous environment for at least 10 minutes.

In some embodiments the carrier content is from about 1% to about 80% by weight, from about 0.5% to about 70% by weight or about 1% to about 60% by weight.

In other embodiments, the carrier is selected from polymer sugars, proteins, long chain hydrophilic block copolymers, reverse phase block copolymers, hyaluronic acid, polyuronic acid, mucopolysaccharide, proteoglycan, polyoxyethylene, surfactants, a polyhydroxy compound, polyhydroxy ester, fatty alcohol, fatty alcohol ester, fatty acid, fatty acid ester, liquid silicone, or mixtures thereof.

In certain embodiments, the demineralized bone matrix also includes an osteoinductive additive selected from bone marrow aspirant, blood, blood products, synthetic and naturally-derived bone morphogenic proteins, growth factors, particulate demineralized bone matrix, or mixtures thereof.

In yet other embodiments, the demineralized bone matrix provided herein includes an osteoconductive additive selected from the group consisting of calcium phosphates, collagen, collagen-derivatives, calcium sulfate, particulate demineralized bone matrix, naturally-derived allogenic bone mineral, naturally-derived autogenic bone mineral or mixtures thereof.

In various embodiments, the demineralized bone matrix prepared from the elongated bone fibers can be delivered in a polymer mesh package.

While multiple embodiments are disclosed, still other embodiments of the present disclosure will become apparent to those skilled in the art from the following detailed description, which shows and describes illustrative embodiments of the disclosure. As will be realized, the various embodiments of the present disclosure are capable of modifications in various obvious aspects, all without departing from the spirit and scope of the present disclosure. Accordingly, the drawing and detailed description are to be regarded as illustrative in nature and not restrictive.

### BRIEF DESCRIPTION OF THE DRAWINGS

FIG. 1 is a flow chart illustrating a method of using an osteobiologic milling machine to produce fibers of the current application.

### DETAILED DESCRIPTION

### Definitions

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Similarly, when values are expressed as approximations, by use of the antecedent “about,” it will be understood that the particular value forms another embodiment that is +/−10% of the recited value. Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value.

Notwithstanding that the numerical ranges and parameters setting forth the broad scope of this application are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

Bioactive agent or bioactive compound is used herein to refer to a compound or entity that alters, inhibits, activates, or otherwise affects biological or chemical events. For example, bioactive agents may include, but are not limited to, osteogenic or chondrogenic proteins or peptides, anti-AIDS substances, anti-cancer substances, antibiotics, immunosuppressants, anti-viral substances, enzyme inhibitors, hormones, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and antiadhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, angiogenic factors, anti-secretory factors, anticoagulants and/or antithrombotic agents, local anesthetics, ophthalmics, prostaglandins, anti-depressants, anti-psychotic substances, anti-emetics, and imaging agents. In certain embodiments, the bioactive agent is a drug. Bioactive agents further include RNAs, such as siRNA, and osteoclast stimulating factors. In some embodiments, the bioactive agent may be a factor that stops, removes, or reduces the activity of bone growth inhibitors. In some embodiments, the bioactive agent is a growth factor, cytokine, extracellular matrix molecule or a fragment or derivative thereof, for example, a cell attachment sequence such as RGD. A more complete listing of bioactive agents and specific drugs suitable for use in the present application may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, edited by Susan Budavari et al., CRC Press, 1996; and the United States Pharmacopeia-25/National Formulary-20, published by the United States Pharmacopeia Convention, Inc., Rockville Md., 2001, each of which is incorporated herein by reference.

Biocompatible, as used herein, is intended to describe materials that, upon administration in vivo, do not induce undesirable long-term effects.

Bone, as used herein, refers to bone that is cortical, cancellous or cortico-cancellous of autogenous, allogenic, xenogenic, or transgenic origin.

Cohesive, as used herein, refers to the ability of the implantable composition of this application to be shaped or packed into a coherent mass which retains its shape and volume and resists erosion from the implant site.

Cohesiveness as used herein refers to the ability of DBM, when mixed with a biocompatible fluid, to form a malleable or flowable mass and to maintain its shape without loss of mass. A demineralized bone mixture is deemed cohesive if greater than 90% of its initial mass and volume are retained within its initial shape dimension in an aqueous environment for at least 10 minutes.

Demineralized, as used herein, refers to any material generated by removing mineral material from tissue, for example, bone tissue. In certain embodiments, the demineralized compositions described herein include preparations containing less than 5% calcium. In some embodiments, the demineralized compositions may comprise less than 1% calcium by weight. Partially demineralized bone is intended to refer to preparations with greater than 5% calcium by weight but containing less than 100% of the original starting amount of calcium. In some embodiments, demineralized bone has less than 95% of its original mineral content. “Demineralized” is intended to encompass such expressions as “substantially demineralized,” “partially demineralized,” “surface demineralized,” and “fully demineralized.” “Partially demineralized” is intended to encompass “surface demineralized.”

Demineralized bone activity refers to the osteoinductive activity of demineralized bone.

Demineralized bone matrix (DBM), as used herein, refers to any material generated by removing mineral material from bone tissue. In some embodiments, the DBM compositions as used herein include preparations containing less than 5% calcium and, in some embodiments, less than 1% calcium by weight. In other embodiments, the DBM compositions comprise partially demineralized bone (e.g., preparations with greater than 5% calcium by weight but containing less than 100% of the original starting amount of calcium).

Osteoconductive, as used herein, refers to the ability of a substance to serve as a template or substance along which bone may grow.

Osteogenic, as used herein, refers to materials containing living cells capable of differentiation into bone tissue.

Osteoinductive, as used herein, refers to the quality of being able to recruit cells from the host that have the potential to stimulate new bone formation. Any material that can induce the formation of ectopic bone in the soft tissue of an animal is considered osteoinductive. For example, most osteoinductive materials induce bone formation in athymic rats when assayed according to the method of Edwards et al., “Osteoinduction of Human Demineralized Bone: Characterization in a Rat Model,” Clinical Orthopaedics & Rel. Res., 357:219-228, December 1998, incorporated herein by reference.

The expression “average length to average thickness ratio” as applied to the DBM elongated fibers of the present application means the ratio of the longest average dimension of the fiber (average length) to its shortest average dimension (average thickness). This is also referred to as the “aspect ratio” of the fiber.

Fibrous, as used herein, refers to bone elements whose average length to average thickness ratio or aspect ratio of the fiber is from about 50:1 to about 1000:1. In overall appearance the fibrous bone elements can be described as elongated bone fibers, threads, narrow strips, or thin sheets. Often, where thin sheets are produced, their edges tend to curl up toward each other. The fibrous bone elements can be substantially linear in appearance or they can be coiled to resemble springs. In some embodiments, the elongated bone fibers are of irregular shapes including, for example, linear, serpentine or curved shapes. The elongated bone fibers are preferably demineralized however some of the original mineral content may be retained when desirable for a particular embodiment.

Non-fibrous, as used herein, refers to elements that have an average width substantially larger than the average thickness of the fibrous bone element or aspect ratio of less than from about 50:1 to about 1000:1. In some embodiments, the non-fibrous bone elements are shaped in a substantially regular manner or specific configuration, for example, triangular prism, sphere, cube, cylinder and other regular shapes. By contrast, particles such as chips, shards, or powders possess irregular or random geometries. It should be understood that some variation in dimension will occur in the production of the elements of this application and elements demonstrating such variability in dimension are within the scope of this application and are intended to be understood herein as being within the boundaries established by the expressions “mostly irregular” and “mostly regular”.

Elongated Bone Fibers.

The elongate bone fibers employed in the implantable compositions of this application are generally characterized as having relatively high average length to average width ratios, also known as the aspect ratio. In various embodiments, the aspect ratio of the elongated bone fibers is at least from about 50:1 to about at least about 1000:1. Such elongated bone fibers can be readily obtained by any one of several methods, for example, by milling or shaving the surface of an entire bone or relatively large section of bone. Thereafter, the resulting elongate bone fibers can be optionally demineralized as discussed herein.

In some embodiments, by employing a milling technique and the milling apparatus described in U.S. Provisional Patent Application No. 61/426,104 filed Dec. 22, 2010 incorporated herein by reference as if set forth in full, elongated bone fibers ranging in average length from about 2 cm up to about 12 cm or more (as in the case of the long bones), and in average width from about 20 mm to about 1 cm can be readily obtained. In some embodiments, the elongated bone fibers can also possess an average length from about 2.5 cm to about 6.0 cm and an average width from about 15 mm to about 50 mm.

In other embodiments, the length of the fibers can be at least about 3.5 cm and average width from about 20 mm to about 1 cm. In various embodiments, the average length of the elongated fibers can be from about 3.5 cm to about 6.0 cm and the average width from about 20 mm to about 1 cm. In other embodiments, the elongated fibers can have an average length be from about 4.0 cm to about 6.0 cm and an average width from about 20 mm to about 1 cm.

In yet other embodiments, the diameter or average width of the elongated fibers is, for example, not more than about 1.00 cm, not more than 0.5 cm or not more than about 0.01 cm. In still other embodiments, the diameter or average width of the fibers can be from about 0.01 cm to about 0.4 cm or from about 0.02 cm to about 0.3 cm.

In another embodiment, the aspect ratio of the fibers can be from about 50:1 to about 950:1, from about 50:1 to about 750:1, from about 50:1 to about 500:1, from about 50:1 to about 250:1; or from about 50:1 to about 100:1. Fibers according to this disclosure can advantageously have an aspect ratio from about 50:1 to about 1000:1, from about 50:1 to about 950:1, from about 50:1 to about 750:1, from about 50:1 to about 600:1, from about 50:1 to about 350:1, from about 50:1 to about 200:1, from about 50:1 to about 100:1, or from about 50:1 to about 75:1.

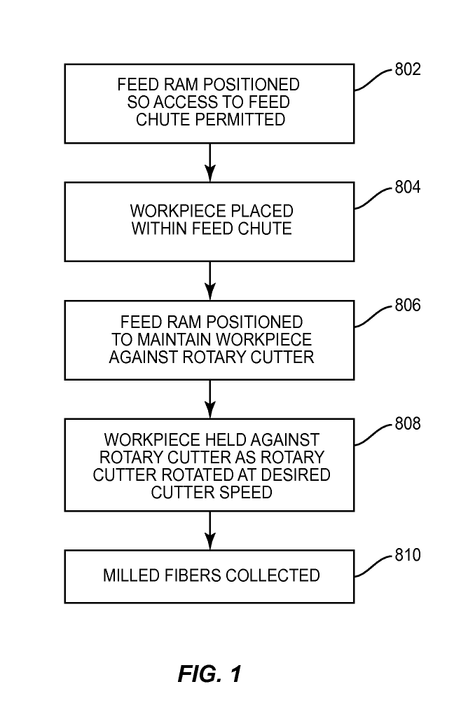

FIG. 1 herein describes a novel method of milling cortical bone, wherein almost 100% of the bone is milled thereby drastically reducing bone waste while at the same time providing elongated bone fibers of increased length, osteoconductivity, flexural and tensile modulus, flexural, tensile and shear strength. The apparatus utilized in the method of milling cortical bone is described in U.S. Provisional Patent Application No. 61/426,104, incorporated herein by reference as if set forth in full.

In FIG. 1, in step 802, the feed ram may be positioned such that access to the feed chute from the access opening is available. In step 804, a workpiece W may be placed within the feed chute from the access opening. A workpiece may be any suitable size and shape that fits within the feed chute. In one embodiment, the workpiece W may be bone, including but not limited to, human donor bone. In step 806, the feed ram may be repositioned to assist in maintaining the workpiece W against the rotary cutter, and in some embodiments, assisting in preventing the workpiece from rotating while it is in contact with the rotary cutter. The force applied to the workpiece W by the feed ram may be provided in any of the manners previously discussed, such as but not limited to, using the forces of gravity on the feed ram, with or without the assistance of selectable angular positioning, using a tightening device, such as a manual crank or drive system, using a screw driver, or using a pneumatic or hydraulic ram. In step 808, the workpiece W may be held against the rotary cutter as the rotary cutter is rotated at a desired cutter speed, such that fibers are milled from the workpiece. In step 810, the fibers may be collected and/or removed from the milling device and used as is or for later processing.

Utilizing the milling apparatus described in U.S. Provisional Patent Application No. 61/426,104 to provide the fibers comprised in the implantable composition of this application up to about one hundred percent (100%) of the workpiece may be successfully milled. A bone material composition comprising the elongated fibers of the present disclosure are prepared by a method of milling fibers comprising inserting a workpiece into a milling apparatus comprising a cutter housing having a feed chute; a rotary cutter, at least partially housed within the cutter housing and in communication with the feed chute; and a feed ram removably positioned within the feed chute for maintaining the workpiece against the rotary cutter; and selectively positioning the feed chute and feed ram at one of a plurality of angular positions with respect to the rotary cutter, such that the force applied by the feed ram on the workpiece is a function of the weight of the feed ram and the angular position of the feed ram with respect to the rotary cutter, wherein the workpiece is a bone, in one aspect a demineralized bone.

In another embodiment, an implantable composition including the elongated fibers described herein is provided, wherein the elongated fibers are prepared by a method of milling fibers comprising inserting a workpiece into a milling apparatus including a cutter housing having a feed chute; a rotary cutter, at least partially housed within the cutter housing and in communication with the feed chute; and a feed ram removably positioned within the feed chute for maintaining the workpiece against the rotary cutter; and selectively positioning the feed chute and feed ram at one of a plurality of angular positions with respect to the rotary cutter, such that the force applied by the feed ram on the workpiece is a function of the weight of the feed ram and the angular position of the feed ram with respect to the rotary cutter.

Providing Demineralized Bone

Following shaving, milling or other technique whereby they are obtained, the elongated fibers are subjected to demineralization in order to reduce their inorganic content to a very low level, in some embodiments, to not more than about 5% by weight of residual calcium and preferably to not more than about 1% by weight residual calcium. Demineralization of the elongated fibers ordinarily results in their contraction to some extent.

Demineralization of the elongated fibers can be conducted in accordance with known conventional procedures. For example, in a demineralization procedure, the elongated fibers useful for the implantable composition of this application are subjected to an acid demineralization step that is followed by a defatting/disinfecting step. The bone is immersed in acid over time to effect its demineralization. Acids which can be employed in this step include inorganic acids such as hydrochloric acid and organic acids such as peracetic acid. After acid treatment, the bone is rinsed with sterile water for injection, buffered with a buffering agent to a final predetermined pH and then finally rinsed with water for injection to remove residual amounts of acid and buffering agent or washed with water to remove residual acid and thereby raise the pH. Following demineralization, the bone is immersed in solution to effect its defatting. An embodiment of defatting/disinfectant solution is an aqueous solution of ethanol, the ethanol being a good solvent for lipids and the water being a good hydrophilic carrier to enable the solution to penetrate more deeply into the bone. The aqueous ethanol solution also disinfects the bone by killing vegetative microorganisms and viruses. Ordinarily at least about 10 to 40 weight percent by weight of water (i.e., about 60 to 90 weight percent of defatting agent such as alcohol) should be present in the defatting/disinfecting solution to produce optimal lipid removal and disinfection within the shortest period of time. In some embodiments, the concentration range of the defatting solution is from about 60 to 85 weight percent alcohol or about 70 weight percent alcohol. Further in accordance with this application, the demineralized elongated bone fibers can be used immediately for preparation of the implant composition or they can be stored under aseptic conditions, advantageously in a lyophilized state prior to such preparation. In another embodiment, the fibrous bone elements can retain some of their original mineral content such that the composition is rendered capable of being imaged utilizing radiographic techniques.

In one embodiment, the demineralized bone is sourced from bovine or human bone. In another embodiment, demineralized bone is sourced from human bone. In one embodiment, the demineralized bone is sourced from the patient's own bone (autogenous bone). In another embodiment, the demineralized bone is sourced from a different animal (including a cadaver) of the same species (allograft bone).

Demineralized Bone Matrix

In various embodiments, this application also provides bone matrix compositions and, more specifically, bone matrix compositions including elongated demineralized bone fibers having an average length greater than at least 2 cm. In various embodiments, the average length of the demineralized bone fibers is from about 2.1 cm to about 6 cm.

To prepare the osteogenic composition utilizing the elongated fibers described herein, a quantity of elongated fibers is combined with a biocompatible carrier to provide a demineralized bone matrix.

Carrier

Generally, materials for the carrier may be biocompatible in vivo and optionally biodegradable. In some uses, the carrier acts as a temporary scaffold until replaced completely by new bone. Suitable carriers can be any number of compounds and/or polymers, such as polymer sugars, proteins, long chain hydrophilic block copolymers, reverse phase block copolymers, hyaluronic acid, polyuronic acid, mucopolysaccharide, proteoglycan, polyoxyethylene, surfactants, including the pluronics series of nonionic surfactants, and peptide thickener. Suggested classes of biocompatible fluid carrier would include polyhydroxy compound, polyhydroxy ester, fatty alcohol (e.g., glycerol), fatty alcohol ester, fatty acid, fatty acid ester, liquid silicone, mixtures thereof, or the like. Settable materials may be used, and they may set up either in situ, or prior to implantation. The bone fibers and carrier (or delivery or support system) together form an osteoimplant useful in clinical applications.

Examples of suitable biocompatible fluid carrier include, but are not limited to:

(i) Polyhydroxy compound, for example, such classes of compounds as the acyclic polyhydric alcohols, non-reducing sugars, sugar alcohols, sugar acids, monosaccarides, disaccharides, water-soluble or water dispersible oligosaccarides, polysaccarides and known derivatives of the foregoing. Specific polyhydroxy compounds include, 1,2-propanediol, glycerol, 1,4-butylene glycol trimethylolethane, trimethylolpropane, erythritol, pentaerythritol, ethylene glycols, diethylene glycol, triethylene glycol, tetraethylene glycol, propylene glycol, dipropylene glycol; polyoxyethylene-polyoxypropylene copolymer, for example, of the type known and commercially available under the trade names Pluronic and Emkalyx; polyoxyethylene-polyoxypropylene block copolymer, for example, of the type known and commercially available under the trade name Poloxamer; alkylphenolhydroxypolyoxyethylene, for example, of the type known and commercially available under the trade name Triton, polyoxyalkylene glycols such as the polyethylene glycols, xylitol, sorbitol, mannitol, dulcitol, arabinose, xylose, ribose, adonitol, arabitol, inositol, fructose, galactose, glucose, mannose, sorbose, sucrose, maltose, lactose, maltitol, lactitol, stachyose, maltopentaose, cyclomaltohexaose, carrageenan, agar, dextran, alginic acid, guar gum, gum tragacanth, locust bean gum, gum arabic, xanthan gum, amylose, mixtures of any of the foregoing, and the like.

(ii) Polyhydroxy ester, for example, liquid and solid monoesters and diesters of glycerol can be used to good effect, the solid esters being dissolved up in a suitable vehicle, for example, propylene glycol, glycerol, polyethylene glycol of 200-1000 molecular weight. Liquid glycerol esters include monacetin and diacetin and solid glycerol esters include such fatty acid monoesters of glycerol as glycerol monolaurate, glyceryl monopalmitate, glyceryl monostearate. In various embodiments, the carrier herein comprises glyceryl monolaurate dissolved in glycerol or a 4:1 to 1:4 weight mixtures of glycerol and propylene glycol, poly (oxyalkylene) glycol ester, or the like.

(iii) Fatty alcohol, for example primary alcohols, usually straight chain having from 6 to 13 carbon atoms, including caproic alcohol, caprylic alcohol, undecyl alcohol, lauryl alcohol, and tridecanol.

(iv) Fatty alcohol ester, for example, ethyl hexyl palmitate, isodecyl neopentate, octadodecyl benzoate, diethyl hexyl maleate, or the like.

(v) Fatty acid having from 6 to 11 carbon atoms, for example, hexanoic acid, heptanoic acid, octanoic acid, decanoic acid and undecanoic acid.

(vi) Fatty acid ester, for example, polyoxyethylene-sorbitan-fatty acid esters, for example, mono- and tri-lauryl, palmityl, stearyl, and oleyl esters including of the type available under the trade name Tween from Imperial Chemical Industries; polyoxyethylene fatty acid esters including polyoxyethylene stearic acid esters of the type known and commercially available under the trade name Myrj; propylene glycol mono- and di-fatty acid esters such as propylene glycol dicaprylate; propylene glycol dilaurate, propylene glycol hydroxy stearate, propylene glycol isostearate, propylene glycol laureate, propylene glycol ricinoleate, propylene glycol stearate, and propylene glycol caprylic-capric acid diester available under the trade name Miglyol; mono-, di-, and mono/di-glycerides, such as the esterification products of caprylic or caproic acid with glycerol, for example, of the type known and commercially available under the trade name Imwitor; sorbitan fatty acid esters, or of the type known and commercially available under the trade name Span, including sorbitan-monolauryl, -monopalmityl, -monostearyl, -tristearyl, -monooleyl and triolcylesters; monoglycerides, for example, glycerol monooleate, glycerol monopalmitate and glycerol monostearate, for example as known and commercially available under the trade names Myvatex, Myvaplex and Myverol, and acetylated, for example, mono- and di-acetylated monoglycerides, for example, as known and commercially available under the trade name Myvacet; isobutyl tallowate, n-butylstearate, n-butyl oleate, or n-propyl oleate.

(vii) Liquid silicone, for example, polyalkyl siloxanes such as polymethyl siloxane and poly (dimethyl siloxane) and polyalkyl arylsiloxane.

In some embodiments of the implantable composition of this application, the liquid carrier is a liquid polyhydroxy compound, liquid polyhydroxy compound derivative, liquid solution of solid polyhydroxy compound, liquid solution of solid polyhydroxy compound derivative or mixtures thereof. If necessary or desirable, in some embodiments, the liquid carrier can be dissolved or diluted with an appropriate solvent such that when combined with the elongated demineralized bone fibers described herein a composition capable of being shaped or packed into a coherent mass which retains its shape and volume over the relatively long term, until the bone formation and remodeling process is completed, is provided. Thus, the polyhydroxy compound or polyhydroxy derivatives can be a liquid in the pure or highly concentrated state at ambient temperature, from about 15° C. to about 50° C., or it can be a solid or semi-solid at this temperature in which case it becomes necessary to dissolve the material in a solvent such as water, physiological saline, ethanol, glycerol, glucose, propylene glycol, polyethylene glycol of from 200-1000 molecular weight, or polyvinyl alcohol. In other embodiments, the liquid carrier can be made up of one or more liquid polyhydroxy compounds or derivatives in solution with one or more solid polyhdroxy compounds or derivatives.

The osteoinductive or biologically active composition may be configured to be moldable, extrudable, or substantially solid. The osteoinductive or biologically active composition may be configured to substantially retain its shape in water for a period of time. The osteoinductive or biologically active composition may form an osteoimplant useful in clinical applications. Suitable carriers may include surface demineralized bone; mineralized bone; nondemineralized cancellous scaffolds; demineralized cancellous scaffolds; cancellous chips; particulate, demineralized, guanidine extracted, species-specific (allogenic) bone; specially treated particulate, protein extracted, demineralized, xenogenic bone; collagen; synthetic hydroxyapatites; synthetic calcium phosphate materials; tricalcium phosphate, sintered hydroxyapatite, settable hydroxyapatite; polylactide polymers; polyglycolide polymers, polylactide-co-glycolide copolymers; tyrosine polycarbonate; calcium sulfate; collagen sheets; settable calcium phosphate; polymeric cements; settable poly vinyl alcohols, polyurethanes; resorbable polymers; and other large polymers; liquid settable polymers; and other biocompatible settable materials. The carrier may further comprise a polyol (including glycerol or other polyhydroxy compound), a polysaccharide (including starches), a hydrogel (including alginate, chitosan, dextran, pluronics, N,O-carboxymethylchitosan glucosamine (NOCC)), hydrolyzed cellulose, or a polymer (including polyethylene glycol). In embodiments wherein chitosan is used as a carrier, the chitosan may be dissolved using known methods including in water, in mildly acidic aqueous solutions, in acidic solutions.

The carrier may further comprise a hydrogel such as hyaluronic acid, dextran, pluronic block copolymers of polyethylene oxide and polypropylene, and others. Suitable polyhydroxy compounds include such classes of compounds as acyclic polyhydric alcohols, non-reducing sugars, sugar alcohols, sugar acids, monosaccharides, disaccharides, water-soluble or water dispersible oligosaccharides, polysaccharides and known derivatives of the foregoing. An example carrier comprises glyceryl monolaurate dissolved in glycerol or a 4:1 to 1:4 weight mixture of glycerol and propylene glycol. Settable materials may be used, and they may set up either in situ, or prior to implantation. Optionally, xenogenic bone powder carriers also may be treated with proteases such as trypsin. Xenogenic carriers may be treated with one or more fibril modifying agents to increase the intraparticle intrusion volume (porosity) and surface area. Useful agents include solvents such as dichloromethane, trichloroacetic acid, acetonitrile and acids such as trifluoroacetic acid and hydrogen fluoride. The choice of carrier may depend on the desired characteristics of the composition. In some embodiments, a lubricant, such as water, glycerol, or polyethylene glycol may be added.

Any suitable shape, size, and porosity of carrier may be used. In some embodiments, the carrier may be settable and/or injectable. Such carrier may be, for example, a polymeric cement, a suitable settable calcium phosphate, a settable poly vinyl alcohol, a polyurethane, or a liquid settable polymer. Hydrogel carriers may additionally impart improved spatial properties, such as handling and packing properties, to the osteoconductive composition. An injectable carrier may be desirable where the composition is used with a containment device. In addition, selected materials must be biocompatible in vivo and optionally biodegradable. In some uses, the carrier acts as a temporary scaffold until replaced by new bone. Polylactic acid (PLA), polyglycolic acid (PGA), and various combinations have different dissolution rates in vivo. In bone, the dissolution rates can vary according to whether the composition is placed in cortical or trabecular bone.

In certain embodiments, the carrier may comprise a shape-retaining solid made of loosely adhered particulate material with collagen. It may alternatively comprise a molded, porous solid, a monolithic solid, or an aggregate of close-packed particles held in place by surrounding tissue. Masticated muscle or other tissue may also be used. Large allogenic bone implants may act as a carrier, for example where their marrow cavities are cleaned and packed with DBM and, optionally, the osteoinductive factors.

In various embodiments, the carrier comprises an osteoinductive material such as a mineralized particulated material, osteoinductive growth factors, or partially demineralized bone. The mineralized particulated material may be TCP, hydroxyapatite, mineral recovered from bone, cancellous chips, cortical chips, surface demineralized bone, or other material. The osteoinductive material may be combined with a further carrier such as starch or glycerol. Accordingly, in some embodiments, the bone matrix may act as a carrier for the tissue-derived extract.

Where, in a particular implantable composition, the fibrous and/or non-fibrous elements exhibit a tendency to quickly or prematurely separate from the carrier component or to otherwise settle out from the composition such that application of a fairly homogeneous composition is rendered difficult or inconvenient, it can be advantageous to include within the composition an optional substance whose thixotropic characteristics prevent or reduce this tendency. Thus, for example, where the carrier component is glycerol and separation of fibrous and/or non-fibrous bone elements occurs to an excessive extent where a particular application is concerned, a thixotropic agent such as a solution of polyvinyl alcohol, polyvinylpyrrolidone, cellulosic ester such as hydroxypropyl methylcellulose, carboxyl methylcellulose, pectin, food-grade texturizing agent, gelatin, dextran, collagen, starch, hydrolyzed polyacrylonitrile, hydrolyzed polyacrylamide, polyelectrolyte such as polyacrylic acid salt, hydrogels, chitosan, other materials that can suspend the fibrous and/or non-fibrous elements, can be combined with the carrier in an amount sufficient to significantly improve the suspension-keeping characteristics of the composition. Optionally, a small amount of non-fibrous DBM may be present in the composition. In one embodiment, this may be in particle form. In one embodiment the particulate DBM would have >50 μm in diameter. In one embodiment, the non-fibrous DBM comprises less than about 10 weight percent of the composition. In a further embodiment, the non-fibrous DBM comprises less than about 5 weight percent of the composition. In a further embodiment, the non-fibrous DBM comprises less than about 1 weight percent of the composition. As a result of their extended length the elongated fibers obtained as described in FIG. 1 herein or by any other means that can produce fibrous elements having an average length of at least 2 cm up to about 6 cm can entangle easily either separately or as part of a demineralized bone matrix. In some embodiments, the elongated bone fibers of the implantable composition are lightly entangled and in other embodiments the elongated bone fibers are densely entangled.

The longer elongated fibers often interlock and become densely entangled. Although not bound by any particular theory or mode of operation, it is believed that the ability of the elongated fibers to interlock and become entangled with each other is advantageous to the formation of coherent and cohesive DBM compositions. In various embodiments, the fibers may be combed using standard fiber combing techniques known in the art to reduce entanglement in order to obtain elongated fibers which are lightly entangled and as a result are less cohesive.

Preparation of DBM Composition

To prepare a DBM composition according to one or more embodiments of this application, a quantity of demineralized elongated bone fibers prepared as described above is combined with water or any other appropriate, biocompatible liquid to form a smooth, flowable, cohesive paste. The resultant implantable composition may be molded or injected into any desired shape and retains its shape, even when submersed in water, saline, or other aqueous solution. An additional benefit of the elongated DBM fibers is that the resultant paste is injectable through an 18-gauge needle.

The liquid may be any biocompatible liquid, including water, saline solution, buffered solutions, serum, bone marrow aspirant, blood, platelet-rich plasma and the like and mixtures thereof. Some biocompatible liquids suitable for use with the short DBM fibers, such as serum, bone marrow aspirant and blood, additionally contain osteoinductive factors that will promote bone growth at the site to which the composition is applied.

The ability of DBM compositions containing the elongated demineralized fibers of the present application to form a cohesive, flowable mixture when combined with only water or saline distinguishes the inventive compositions from previous DBM compositions, which require viscous carrier liquids such as glycerol, gel, gelatin, hyaluronic acid, or hydrogel polymers or even binders to form a cohesive material. While the elongated fiber DBM compositions of the present application may be formed using aqueous solutions, the compositions are not limited to the use of such aqueous solutions.

Optional Additives

If desired, the fibrous and/or non-fibrous bone elements of this application can be modified in one or more ways. In various embodiments, any of a variety of medically and/or surgically useful optional substances can be incorporated in, or associated with, the bone elements before, during, or after preparation of the implantable composition. Thus, in some embodiments, one or more of such substances can be introduced into the bone elements, for example, by soaking or immersing the bone elements in a solution or dispersion of the desired substance(s), by adding the substance(s) to the carrier component of the implantable composition or by adding the substance(s) directly to the implantable composition.

Medically/surgically useful substances which can be readily combined with the elongated bone fibers, fluid carrier and/or implantable composition of this application include, for example, collagen, insoluble collagen derivatives, hydroxyapatite, and soluble solids and/or liquids dissolved therein, for example, antiviricides, particularly those effective against HIV and hepatitis; antimicrobials and/or antibiotics such as erythromycin, bacitracin, neomycin, penicillin, polymyxin B, tetracyclines, viomycin, chloromycetin and streptomycins, cefazolin, ampicillin, azactam, tobramycin, clindamycin and gentamycin; amino acids, peptides, vitamins, inorganic elements, inorganic compounds, cofactors for protein synthesis, hormones; endocrine tissue or tissue fragments; synthesizers; enzymes such as collagenase, peptidases, oxidases; polymer cell scaffolds with paraenchymal cells; angiogenic drugs and polymeric carriers containing such drugs; collagen lattices; biocompatible surface active agents; antigenic agents; cytoskeletal agents; cartilage fragments, living cells such as chondrocytes, bone marrow cells, mesenchymal stem cells, natural extracts, tissue transplants, bioadhesives, bone morphogenic proteins (BMPs), transforming growth factor (TGF-beta), insulin-like growth factor (IGF-1) (IGF-2), platelet derived growth factor (PDGF), fibroblast growth factors (FGF), vascular endothelial growth factor (VEGF), angiogenic agents, bone promoters, cytokines, interleukins, genetic material, genes encoding bone promoting action, cells containing genes encoding bone promoting action; growth hormones such as somatotropin; bone digestors; antitumor agents; fibronectin; cellular attractants and attachment agents; immuno-suppressants; permeation enhancers, for example, fatty acid esters such as laureate, myristate and stearate monesters of polyethylene glycol, surface active agents, enamine derivatives, α-keto aldehydes; nucleic acids; epidermal growth factor (EGF); all collagen types (not just type 1); non-collagenous proteins such as osteopontin, osteonectine, bone sialo proteins, vitronectin, thrombospondin, proteoglycans, decorin, biglycan, aggrecan, versican, tenascin, matrix gla protein hyaluronan; soluble and insoluble components of the immune system, soluble and insoluble receptors including truncated forms, soluble, insoluble and cell surface bound ligands including truncated forms; chemokines, bioactive compounds that are endocytosed; compounds capable of altering the membrane potential of cells, compounds capable of altering the monovalent and divalent cation/anion channels of cells; bone resorption inhibitors and stimulators; angiogenic and mitogenic factors; bioactive factors that inhibit and stimulate second messenger molecules; integrin adhesion molecules; clotting factors; externally expanded autograft or xenograft cells and any combinations thereof. The amounts of such optionally added substances can vary widely with optimum levels being readily determined in a specific case by routine experimentation.

The demineralized bone matrix prepared with the elongate bone fibers described herein may comprise a number of materials in combination, some or all of which may be in the form of fibers and/or particles. The matrix may comprise calcium phosphates. Driessens et al. “Calcium phosphate bone cements,” Wise, D. L., Ed., Encyclopedic Handbook of Biomaterials and Bioengineering, Part B, Applications New York: Marcel Decker; Elliott, Structure and Chemistry of the Apatites and Other Calcium Phosphates Elsevier, Amsterdam, 1994, each of which is incorporated by reference. Calcium phosphate matrices include, but are not limited to, dicalcium phosphate dihydrate, monetite, tricalcium phosphate, tetracalcium phosphate, hydroxyapatite, nanocrystalline hydroxyapatite, poorly crystalline hydroxyapatite, substituted hydroxyapatite, and calcium deficient hydroxyapatites. In some embodiments, the bone fibers may be added to a carrier.

Implantable DBM compositions have been used for many years in orthopedic medicine to promote the formation of bone. For example, DBM compositions have found use in the repair of fractures, in the fusion of vertebrae, in joint replacement surgery, and in treating bone destruction due to underlying disease such as rheumatoid arthritis. DBM is thought to promote bone formation in vivo by osteoconductive and osteoinductive processes. The osteoinductive effect of implanted DBM compositions is thought to result from the presence of active growth factors present on the isolated collagen-based matrix. These factors include members of the TGF-β, IGF, and BMP protein families. Particular examples of osteoinductive factors include TGF-β, IGF-1, IGF-2, BMP-2, BMP-7, parathyroid hormone (PTH), and angiogenic factors. Other osteoinductive factors such as osteocalcin and osteopontin are also likely to be present in DBM preparations as well. There are also likely to be other unnamed or undiscovered osteoinductive factors present in DBM.

In some embodiments, the demineralized bone may be further treated to affect properties of the bone. For example, the DBM may be treated to disrupt the collagen structure of the DBM. Such treatment may comprise collagenase treatment, heat treatment, mechanical treatment, or other. While demineralized bone is specifically discussed herein, in some embodiments, the teachings herein may be applied to non-demineralized bone, to partially demineralized bone, or to surface demineralized bone.

In some embodiments, biological activities of the bone matrix may be increased. Accordingly, the bone matrix, and compositions formed from the bone matrix, may variously be referred to as biologically active and/or, in some cases, osteoinductive. The biological activities of the bone composition provided herein that may be increased include but are not limited to osteoinductive activity, osteogenic activity, chondrogenic activity, wound healing activity, neurogenic activity, contraction-inducing activity, mitosis-inducing activity, differentiation-inducing activity, chemotactic activity, angiogenic or vasculogenic activity, exocytosis or endocytosis-inducing activity, or other cell or biological activity. It will be appreciated that bone formation processes frequently include a first stage of cartilage formation that creates the basic shape of the bone, which then becomes mineralized (endochondral bone formation). Thus, in many instances, chondrogenesis may be considered an early stage of osteogenesis, though of course it may also occur in other contexts.

In accordance with various embodiments, the bone matrix provided herein may be used with growth factors, extracts, peptide hormones, or other additives to increase the osteoinductive capacity or that otherwise encourage cell or biological activity of the bone matrix or to impart other benefits to the bone matrix. It will be appreciated that the amount of additive used will vary depending upon the type of additive, the specific activity of the particular additive preparation employed, and the intended use of the composition. The desired amount is readily determinable by the user.

Any of a variety of medically and/or surgically useful optional substances can be incorporated in, or associated with, the osteoinductive factors either before, during, or after preparation of the osteoinductive or biologically active composition. Thus, for example when elongated demineralized bone fibers of this application are used to form the material, one or more of such substances may be introduced into the elongated demineralized bone fibers, for example, by soaking or immersing these bone fibers in a solution or dispersion of the desired substance(s).

In one embodiment, a tissue-derived extract may be added to the bone matrix. U.S. patent application Ser. No. 12/140,044 discloses such extracts and addition of such extracts to DBM and is incorporated herein by reference. For example, a tissue-derived extract or partially demineralized bone may be added to the bone matrix. The extract may be derived from any suitable tissue, such as bone, bladder, kidney, brain, skin, or connective tissue. Further, the extract may be derived in any suitable manner. The extract may be allogeneic, autogeneic, xenogeneic, or transgenic. In embodiments wherein the extract is bone-derived, the bone may be cortical, cancellous, or corticocancellous and may be demineralized, partially demineralized, or mineralized. In some embodiments, the extract may comprise demineralized bone, partially demineralized bone, mineral derived from bone, or collagen derived from bone. In some embodiments, the tissue-derived extract may be a protein extract.

Bone regeneration involves a multitude of cells, for example, cartilage, fibroblasts, endothelial cells besides osteoblasts. Accordingly, the bone matrix composition may be used to deliver stem cells, which offers the potential to give rise to different types of cells in the bone repair process. In one embodiment, the bone matrix composition further comprises a cell such as an osteogenic cell or a stem cell.

In various embodiments, the additive may comprise radiopaque substances, angiogenesis promoting materials, bioactive agents, osteoinducing agents, or other. Such materials would include without limitation barium sulfate, iodine-containing compounds, titanium and mineralized bone.

In certain embodiments, the additive is adsorbed to or otherwise associated with the bone matrix. The additive may be associated with the bone matrix through specific or non-specific interactions, or covalent or noncovalent interactions. Examples of specific interactions include those between a ligand and a receptor, an epitope or an antibody. Examples of nonspecific interactions include hydrophobic interactions, electrostatic interactions, magnetic interactions, dipole interactions, van der Waals interactions, or hydrogen bonding. In certain embodiments, the additive is attached to the bone matrix composition, for example, to the carrier, using a linker so that the additive is free to associate with its receptor or site of action in vivo. In other embodiments the additive is either covalently or non-covalently attached to the carrier. In certain embodiments, the additive may be attached to a chemical compound such as a peptide that is recognized by the carrier. In another embodiment, the additive is attached to an antibody, or fragment thereof, that recognizes an epitope found within the carrier. In certain embodiments at least additives are attached to the osteoimplant. In other embodiments at least three additives are attached to the osteoinductive or biologically active composition. An additive may be provided within the osteoinductive or biologically active composition in a sustained release format. For example, the additive may be encapsulated within biodegradable polymer nanospheres, or microspheres.

Flow additives according to this application can include, but are not limited to, small molecule organic compounds, polymeric/oligomeric materials, and solutions thereof. In some embodiments, when added to the implantable composition containing the elongated bone fibers the viscosity thereof should be sufficiently changed to allow flow through a syringe needle of about 8-gauge or greater (greater number gauges of syringe needles have smaller diameters, thus requiring lower threshold viscosity through which they may flow), preferably of about 12-gauge or greater, for example of about 14-gauge or greater, of about 15-gauge or greater, or of about 18-gauge or greater. Sufficient flow can be understood, in terms of syringe needles, to result in an injection force of not more than 50 pounds, preferably not more than 40 pounds. In another embodiment, the flow additive modifies the viscosity of the composition to which it is added such that the composition is capable of flowing through a syringe needle having a gauge size from about 8 to about 18, alternately from about 8 to about 15, from about 12 to about 18, or from about 12 to about 15.

When present, the amount of flow additive that can be added to the composition can be from about 0.01% to about 1.5% by weight of the elongated fiber composition from about 0.1% to about 1% by weight, or from about 0.05% to about 1% by weight. In an alternate embodiment, the amount of flow additive can be from about 1.5% to about 5% by weight of the elongated fiber composition. In a preferred embodiment, the flow additive, when used, is present in an amount of about 0.5% by weight of the composition.

Suitable examples of flow additives can include, but are in no way limited to, hyaluronic acid; hyaluronate salts such as sodium, potassium, lithium, or the like, or a combination thereof; alginate salts such as sodium, potassium, lithium, or the like; starch compounds, which can be present in its natural form, in a destructured form, or in any number of chemically modified derivative forms (for example, alkyoxylated derivatives, esterified derivatives, ionically modified starches, oxidized starches, grafted starches, crosslinked starches, or the like, or mixtures thereof); saturated, monounsaturated, and/or polyunsaturated oils, such as those extracted or isolated from plant and/or animal sources, including, but not limited to, sunflower, safflower, peanut, castor bean, sesame, coconut, soybean, corn, canola, olive, vegetable, palmitins, stearins, oleins, and the like, or derivatives or combinations thereof, as naturally extracted, as synthesized, or as modified or processed in some way, partially or fully hydrogenated, partially or fully dehydrogenated, partially or fully saponified, partially or fully acidified, partially halogenated, or the like; a wax including, but not limited to, hydrocarbon waxes (for example, polyolefin waxes, such as polyethylene wax, polypropylene wax, and the like, or copolymers thereof), oligoester waxes, monoester waxes, oligoether waxes, monoether waxes, and the like, or combinations thereof, as naturally extracted, as synthesized, or as modified or processed in some way, partially or fully hydrogenated, partially or fully dehydrogenated, partially or fully saponified, partially or fully acidified, partially halogenated, or the like; cellulosic compounds, including, but not limited to, native or synthetic cellulose, cotton, regenerated cellulose (for example, rayon, cellophane, or the like), cellulose acetate, cellulose propionate, cellulose butyrate, cellulose acetate-propionate, cellulose acetate-butyrate, cellulose propionate-butyrate, cellulose nitrate, methyl cellulose, ethyl cellulose, carboxymethyl cellulose, carboxyethyl cellulose, cellulose salts, and combinations or copolymers thereof, as naturally extracted, as synthesized, or as modified or processed in some way, including partially or fully esterified, partially or fully nitrated, partially or fully regenerated, partially or fully etherified, partially or fully acidified, partially or fully acid-neutralized, or the like, or combinations thereof; surface-active biomolecules or (co)polymers; poly(ethylene glycol) and/or poly(ethylene oxide) oligomers, homopolymers, or copolymers; autologous substances such as autologous bone marrow aspirates, autologous blood substances, or the like, or a combination thereof; heterologous substances such as allogeneic bone marrow aspirates, xenogenic bone marrow aspirates, allogeneic blood substances, xenogenic blood substances, or the like, or a combination thereof; or the like, or combinations thereof. In a preferred embodiment, the flow additive comprises hyaluronic acid and/or a hyaluronate salt. In another preferred embodiment, the flow additive comprises sodium hyaluronate. In an alternate embodiment, the flow additive can include chondroitin, glucosamine, hyaluronic acid, a salt thereof, or a mixture thereof.

In one or more embodiments, an additive is included in the DBM composition to further modify the handling characteristics of the composition, such as viscosity and moldability. The additive may be a biocompatible polymer, such as a water-soluble cellulosic, or a natural polymer, such as gelatin. The additive may be added to either the dry DBM component or the liquid component. The additive may be used to at least partially coat the DBM fibers prior to combining them with the liquid carrier. Non-limiting examples of additives suitable for use in the DBM composition include gelatin, carboxymethyl cellulose, hydroxypropyl methylcellulose, methylcellulose, hydroxyethyl cellulose, other cellulose derivatives, alginate, hyaluronic acid, sodium salts, polyvinyl pyrrolidones, polyvinyl alcohol, arabic gum, guar gum, xantham gum, chitosans, and poloxamers.

As previously indicated, the implantable composition of this disclosure can be freshly prepared just by mixing desired quantities of the demineralized fibrous bone elements, fluid carrier and optional component(s), if any, in any suitable sequence of separate mixing, adsorption, rehydration or drying operations or all at once. Thus, the demineralized fibrous bone elements can be mixed with the optional ingredients(s) and thereafter combined with the fluid carrier component, the demineralized fibrous bone elements can be mixed with the fluid carrier followed by addition of the optional ingredient(s) or the optional ingredients can be added to the fluid carrier followed by addition of the demineralized fibrous bone elements. Variations of these and other sequences of mixing are, of course, possible. In various embodiments, the implantable composition can include non-fibrous bone elements. In other embodiments, the fibrous elements and fluid carrier are mixed substantially simultaneously such that the fibrous elements of the implantable composition are entangled and the non-fibrous bone elements are thoroughly mixed in the entangled fibrous bone elements.

The elongated fibers disclosed herein are naturally more osteoconductive than non-fibrous elements, as cells, for example, osteoclasts and osteoblasts, can travel along the length of the elongated fiber farther and with greater orientation to gain access to the composite interior of the bone deminerallized matrix. The entangled fiber network provides a continuous pathway for improved cellular access over the elongated fibers of implantable composition utilized in DBM and as a result an improvement in osteoconductivity is, therefore, expected.

The amount of demineralized elongated bone fibers which can be incorporated into the implantable composition can vary widely with amounts of about 99% weight, about 95% by weight, about 90% by weight, about 85% by weight 70% by weight. In various embodiments, the amount of the non-fibrous bone elements which can be incorporated into the implantable composition can vary widely with amounts from about 10 to about 90 weight percent, and preferably from about 20 to about 70 weight percent. The ratio of fibrous to non-fibrous bone elements can vary between about 0.2:1 to about 1:0.2. The balance of the composition being made up of fluid carrier and optional ingredient(s), if any. (2010/0111906)

In various embodiments, the bone matrix provided herein may be combined, with or without additives, with a carrier or excipient to achieve consistency for specific uses. For example, a carrier may be selected to provide the bone matrix composition in a gel consistency, a putty consistency, a paste consistency, or other to form an osteoinductive or biologically active composition. In all instances, the use of the elongated bone fibers disclosed herein results in a DBM having enhanced cohesiveness with and enhanced ability to be shaped and packed into a coherent mass, which retains its shape and volume and resists erosion from the implant site. Moreover, the use of the elongated fibers described herein allow for obtaining a DBM having reduced carrier content. In some embodiments, the carrier content can be from about 1 to about 80% by weight, from about 1 to about 75% by weight, from about 1 to about 70% by weight, from about 1 to about 65% by weight without losing cohesiveness.

The bone matrix composition may be completely insoluble or may be slowly solubilized after implantation. Following implantation, the composition may resorb or degrade, remaining substantially intact for at least one to seven days, or for two or four weeks or longer and often longer than 60 days. The composition may thus be resorbed prior to one week, two weeks, three weeks, or other, permitting the entry of bone healing cells.

Formation of an Implant

The bone matrix compositions provided herein may be used to form an osteoinductive or biologically active osteoimplant. The osteoimplant resulting from the bone matrix, additive, and/or carrier may be flowable, have a putty consistency, may be shaped or molded, and/or may be deformable. The osteoimplant may assume a determined or regular form or configuration such as a sheet, plate, disk, tunnel, cone, or tube, to name but a few. Prefabricated geometry may include, but is not limited to, a crescent apron for single site use, an I-shape to be placed between teeth for intra-bony defects, a rectangular bib for defects involving both the buccal and lingual alveolar ridges, neutralization plates, reconstructive plates, buttress plates, T-buttress plates, spoon plates, clover leaf plates, condylar plates, compression plates, bridge plates, or wave plates. Partial tubular as well as flat plates can be fabricated from the osteoimplant. Such plates may include such conformations as, for example, concave contoured, bowl shaped, or defect shaped. The osteoimplant can be machined or shaped by any suitable mechanical shaping means. Computerized modeling can provide for the intricately-shaped three-dimensional architecture of an osteoimplant custom-fitted to the bone repair site with great precision. In embodiments wherein the osteoimplant is shaped or moldable, as a result of the inclusion of the elongated demineralized bone fibers of this application the implant can retain coherence or cohesiveness in fluids.

In certain embodiments, the osteoinductive or biologically active bone matrix composition may be subjected to a configuring step to form an osteoimplant. The configuring step can be employed using conventional equipment known to those skilled in the art to produce a wide variety of geometries, e.g., concave or convex surfaces, stepped surfaces, cylindrical dowels, wedges, blocks, screws, or the like.

To facilitate on-site preparation and/or usage of the composition herein, the demineralized fibrous bone elements and non-fibrous bone elements, preferably in lyophilized or frozen form, and fluid carrier (the latter containing one or more optional ingredients such as those identified above) can be stored in separate packages or containers under sterile conditions and brought together in intimate admixture at the moment of use for immediate application to an osseous defect site employing any suitable means such as spatula, forceps, syringe, tamping device, and the like. Alternatively, the implant composition can be prepared well in advance and stored under sterile conditions until required for use. When the implant composition is prepared well in advance it is preferably lyophilized prior to packaging for storage. In some embodiments, the composition described herein can be combined with autograft bone marrow aspirate, autograft bone, preparations of selected autograft cells, autograft cells containing genes encoding bone promoting action prior to being placed in a defect site. In various embodiments, the implant composition is packaged already mixed and ready for use in a suitable container, such as for example, syringe, resealable non-toxic bottle, a bag mesh or pouch or is provided as a kit which can be prepared at a surgeon's direction when needed.

In some embodiments, the implantable composition can be delivered within a porous mesh that will provide targeted and contained delivery. The polymer mesh can comprise a polymer (such as polyalkylenes (e.g., polyethylenes, polypropylenes, etc.), polyamides, polyesters, polyurethanes, poly(lactic acid-glycolic acid), poly(lactic acid), poly(glycolic acid), poly(glaxanone), poly(orthoesters), poly(pyrolicacid), poly(phosphazenes), L-co-G, etc.), other bioabsorbable polymer such as Dacron or other known surgical plastics, a natural biologically derived material such as collagen, a ceramic (with bone-growth enhancers, hydroxyapatite, etc.), PEEK (polyether-etherketone), dessicated biodegradable material, metal, composite materials, a biocompatible textile (e.g., cotton, silk, linen), or other. In one embodiment, the containment device is formed as a long bag-like device and may be used with minimally invasive techniques.

The polymer mesh is generally designed for effective cellular in-growth and complete resorption within three to six months, while not interfering with bone regeneration. The polymer mesh provides a controlled environment for proximate interaction of the implantable composition eliminating issues with graft site migration or irrigation that is often seen with currently available bone graft substitutes. The implant composition of this application can be firmly placed into an appropriate size defect site to maintain volume and provide support for adjacent tissues. Such placement can be accomplished through the use of a variety of devices such as, for example, spatula, forceps, syringe, tamping device or delivered within a polymer mesh.

The implant composition of this application can be tailored to be utilized for a variety of orthopedic, neurosurgical, and oral and maxillofacial surgical indications in which it would be advantageous to be able to firmly place the composition into a bone defect site such as the repair of simple and compound fractures and nonunions, external fixations, joint reconstructions such as arthrodesis, general arthroplasty, acetabular repair, cup arthroplasty of the hip, femoral and humeral head replacement, femoral head surface replacement and total joint replacements, repairs of the vertebral column including spinal fusion and internal fixation, tumor surgery, for example, deficit filling, discectomy, laminectomy, excision of spinal cord tumors, anterior cervical and thoracic operations, repair of spinal injuries, scoliosis, lordosis and kyphosis treatments, intermaxillary fixation of fractures, mentoplasty, temporomandibular joint replacement, alveolar ridge augmentation and reconstruction, inlay bone grafts, implant placement and revision, sinus lifts, furcation defects, periodontal defects, dental defects, ulna defects, metaphyseal defects, tibia plateau defects, wrist defects, ankle defects, and the like.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A plurality of elongated bone fibers having an average length from about 2 cm to about 6 cm, the plurality of elongated bone fibers entangled in a carrier matrix.

2. A plurality of elongated bone fibers of claim 1 having an average length from about 2.1 cm to about 6 cm.

3. An implantable composition comprising a plurality of elongated bone fibers having an average length from about 2 cm to about 6 cm, the plurality of elongated bone fibers entangled in a carrier matrix.

4. An implantable composition of claim 3, wherein the fibers have an average length from about 2.1 cm to about 6 cm.

5. An implantable composition of claim 3, wherein the content of the plurality of elongated demineralized bone fibers is about 99% by weight, about 95% by weight, about 90% by weight, or about 80% by weight of a total weight of the composition.

6. An implantable composition of claim 3, wherein the each elongated demineralized bone fiber has an average length greater than its average width.

7. An implantable composition of claim 3, wherein the aspect ratio of each elongated demineralized bone fiber is from about 50:1 to about 1000:1, from about 50:1 to about 950:1, from about 50:1 to about 750:1, from about 50:1 to about 500:1, from about 50:1 to about 250:1; or from about 50:1 to about 100:1.

8. An implantable composition of claim 3, wherein the plurality of elongated demineralized bone fibers is obtained from cortical autogenic, cortical allogenic, cortical xenogenic cancellous autogenic, cancellous allogenic, cancellous xenogenic, cortical transgenic, cancellous transgenic, corticocancellous autogenic, corticocancellous allogenic, corticocancellous xenogenic or corticocancellus transgenic bone.

9. An implantable composition of claim 3 further comprising an additive selected from collagen, collagen derivatives, antiviricides, antimicrobials, antibiotics, biocidal sugars, amino acids, peptides, vitamins, inorganic elements, co-factors for protein synthesis, hormones, endocrine tissue, endocrine tissue fragments, enzymes, polymer cell scaffolds with parenchymal cells, angiogenic drugs, collagen lattices, antigenic agents, cytoskeletal agents, cartilage fragments, living cells, natural extracts, tissue transplants, demineralized bone powder, autogenous tissues, bioadhesives, bone morphogenic proteins (BMPs), angiogenic factors, transforming growth factor (TGF-beta), insulin-like growth factor (IGF-1), growth hormones, bone digestors, antitumor agents, immuno-suppressants, permeation enhancers, enamine derivatives, nucleic acids or combinations thereof.

10. An implantable composition of claim 3 further comprising at least one radiopaque material selected from barium sulfate, iodine containing compounds, titanium, mineralized bone or mixtures thereof.

11. An implantable composition of claim 3 further comprising at least one additive selected from stem cells, autograft bone marrow aspirate, autograft bone, preparations of selected autograft cells, autograft cells containing genes encoding bone promoting action, autograft cells expanded outside the body and returned or combinations thereof.

12. A demineralized bone matrix composition comprising a plurality of elongated demineralized bone fibers having an average length of from about 2 cm to about 6 cm entangled in a carrier in an amount sufficient to produce a cohesive formable mass.

13. A demineralized bone matrix composition of claim 12, wherein greater than 90% of the cohesive formable mass retains its initial shape dimension in an aqueous environment for at least 10 minutes.

14. A demineralized bone matrix composition of claim 12, wherein the elongated demineralized bone fibers have an average length that is greater than its average width and each fiber is in a range from about 2.0 cm to about 6 cm.

15. A demineralized bone matrix composition of claim 12, wherein the carrier content is from about 1% to about 90% by weight, from about 1% to about 85% by weight or about 1% to about 80% by weight.

16. A demineralized bone matrix composition of claim 12, wherein the carrier comprises polymer sugars, proteins, long chain hydrophilic block copolymers, reverse phase block copolymers, hyaluronic acid, polyuronic acid, mucopolysaccharide, proteoglycan, polyoxyethylene, surfactants, a polyhydroxy compound, polyhydroxy ester, fatty alcohol, fatty alcohol ester, fatty acid, fatty acid ester, liquid silicone, or mixtures thereof.

17. A demineralized bone matrix composition of claim 12 further comprising additives, which comprise bioactive compounds, growth factors, extracts, peptide hormones, antiviricides, inorganic compounds, cofactors for protein synthesis, endocrine tissue, enzymes, angiogenic drugs and polymeric carriers containing such drugs, collagen lattice, biocompatible surface active agents; antigenic agents, cytoskeletal agents, cartilage fragments, living cells, tissue transplants, bioadhesives, bone morphogenic proteins (BMPs), transforming growth factor (TGF-beta), insulin-like growth factor (IGF-1) (IGF-2), platelet derived growth factor (PDGF), fibroblast growth factors (FGF), vascular endothelial growth factor (VEGF), angiogenic agents, bone promoters, cytokines, interleukins, genetic material, genes encoding bone promoting action, stem cells, cells containing genes encoding bone promoting action, antitumor agents, fibronectin, immuno-suppressants, nucleic acids, epidermal growth factor (EGF), collagen, non-collagenous proteins bone resorption inhibitors and stimulators, angiogenic and mitogenic factors, bioactive factors, integrin adhesion molecules, clotting factors, externally expanded autograft, externally expanded xenograft cells or any combinations thereof.

18. A demineralized bone matrix composition of claim 12 further comprising an osteoinductive additive comprising bone marrow aspirant, blood, blood products, synthetic and naturally-derived bone morphogenic proteins, growth factors, particulate demineralized bone matrix, or mixtures thereof.

19. A demineralized bone matrix composition of claim 12 further comprising an osteoconductive additive comprising calcium phosphates, collagen, collagen-derivatives, calcium sulfate, particulate demineralized bone matrix, naturally-derived allogenic bone mineral, naturally-derived autogenic bone mineral or mixtures thereof.

20. A demineralized bone matrix composition of claim 12 contained in a polymer mesh.
